(19) United States
(12) Patent Application Publication
(10) Pub. No.: US 2007/0243248 A1
(43) Pub. Date: Oct. 18, 2007

(54) Rapidly disintegrating solid oral dosage form of liquid dispersions

(76) Inventor: S. Rao Cherukuri, Vienna, VA (US)

Correspondence Address: THORPE NORTH & WESTERN, LLP., 8180 SOUTH 700 EAST, SUITE 350, SANDY, UT, 84070, US

(21) Appl. No.: 11/787,115

(22) Filed: Apr. 12, 2007

Related U.S. Application Data
(60) Provisional application No. 60/744,845, filed on Apr. 14, 2006.

(51) Int. Cl. A61K 9/20

(52) U.S. Cl. 424/464

(57) ABSTRACT

Solid dose rapidly disintegrating compositions for administering pharmaceutical and nutritional supplement agents and methods for the preparation thereof are disclosed and described. Preparation methods which maintain the particulate size of an active agent at the pre-processing size in the final composition are further disclosed. The ability to maintain such particulate size provides a number of advantages, including improved bioavailability and more accurate dosing.

### PRIORITY DATA

This application claims the benefit of U.S. Provisional Patent Application Ser. No. 60/744,845, filed on Apr. 14, 2006, which is incorporated herein by reference.

### FIELD OF THE INVENTION

Rapidly disintegrating solid oral dosage forms are disclosed which comprise an active ingredient and at least one pharmaceutically acceptable liquid volume reduction agent wherein the liquid volume reduction agent comprises from about 10% to about 90% w/w of the composition. Methods for preparing and using said dosage forms are also presented.

### BACKGROUND OF THE INVENTION

Rapidly disintegrating dosage forms are known in the art. See for example, U.S. Pat. No. 5,607,697, which describes a solid dosage form consisting of coated microparticles that disintegrate in the mouth. The microparticle core has a pharmaceutical agent and the core is coated with a material that retards dissolution in the mouth and masks the taste of the pharmaceutical agent. The microparticles are then compressed to form a tablet. Other excipients can also be added to the tablet formulation. See also, U.S. Pat. Nos. 5,503,846, 5,223,264; 5,178,878; 5,401,513; and 5,219,574. Other publications include: U.S. Pat. Nos. 5,871,781; 5,869,098; 5,866,163; 5,851,553; 5,622,719; 5,567,439; 5,587,172; 5,464,632; 4,642,903 5,188,825; 5,631,023; 5,827,541; 5,576,014; 5,446,464; 5,807,576; 5,635,210; 5,595,761; 5,587,180; 5,776,491; 5,639,475; 5,709,886; 5,807,578; 5,807,577; 5,112,616; 5,073,374, which are all incorporated by reference.

### SUMMARY OF THE INVENTION

Novel rapidly disintegrating solid dose oral formulations of liquid dispersions of active agents are presented. These formulations provide easy dosage form administration and consumer convenience and compliance, fast onset of therapeutic activity combined with substantially complete disintegration of the formulation in less than about one minute.

In another aspect, there is provided a method of preparing rapidly disintegrating solid dose oral formulations. The method comprises: (1) providing a liquid dispersion of an active agent; (2) adding at least one pharmaceutically acceptable liquid volume reduction agent; (3) forming a semi-solid viscous composition; and (4) forming rapidly disintegrating particles. The methods further comprise processing the rapidly disintegrating particles into rapidly disintegrating dosage forms such as powders, granules, tablets, etc. suitable for oral administration. Additional pharmaceutically acceptable excipients can also be added to the composition for administration.

Yet another aspect of the present invention provides a method of treating a mammal, including a human, requiring rapid onset of therapeutic activity with a rapidly disintegrating composition of the invention.

It is to be understood that both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

### DEFINITIONS

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of drugs or active agents, and reference to “the carrier” includes reference to one or more of such carriers.

As used herein, “substantially” refers to situations close to and including 100%. Substantially is used to indicate that, though 100% is desirable, a small deviation therefrom is acceptable. For example, substantially all asperities includes groups of all asperities and groups of all asperities minus a relatively small portion of asperities.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

### THE INVENTION

Rapidly disintegrating dosage forms disintegrate or disperse rapidly in a biological medium such as water, saliva, tears, vaginal fluids, or plasma or on the skin or mucosal surface. For example, when the dosage form is administered orally, the dosage form disintegrates or disperses rapidly the patient's mouth without chewing or the need for water within a very short time. Rapidly dissolving dosage forms can be beneficial because of their ease of administration and convenience.

The rapidly disintegrating formulations of the invention comprise an active agent to be administered, at least one liquid volume reduction agent from about 15% to about 90% by weight, and optionally other pharmaceutically acceptable excipients such as diluents, lubricants, binders, disintegrants, flavorants, colorants, etc. The active agent can be in a crystalline form, semi-crystalline form, amorphous form, or a combination thereof. The active agent may be a poorly soluble drug or a fairly soluble drug.

A rapidly disintegrating solid oral dosage form according to the invention has a disintegration time of less than about 1 minute upon addition to an aqueous medium. More preferably, the dosage form has a disintegration time upon addition to an aqueous medium of less than about 45 seconds, less than about 30 seconds, less than about 20 seconds, less than about 15 seconds, less than about 10 seconds, or less than about 5 seconds.

The rapidly disintegrating solid dose compositions can be formulated to mask the unpleasant taste of an active agent. Such taste masking can be accomplished, for example, by the addition of one or more sweet tasting excipients, by coating the active agent with a sweet tasting excipient, and/or by coating a dosage form of the active agent, and excipients with a sweet tasting excipient.

The rapidly disintegrating solid oral dosage forms may also comprise nanoparticulate or nanoemulsion dispersions. For example, nanoparticulate compositions, first described in U.S. Pat. No. 5,145,684 (“the '684 patent”), may consist of a particles of poorly soluble active agent having adsorbed onto the surface thereof a non-crosslinked surface stabilizer. The '684 patent also describes methods of making such nanoparticulate compositions, which is incorporated by reference herein. Other publications relating to nanoparticulate compositions include: U.S. Pat. Nos. 5,518,187; 5,862,999; 5,718,388; 5,510,118; 5,318,767; 5,399,363; 5,494,683; 5,429,824; 5,518,738; 5,552,160; 5,747,001, all of which are incorporated by reference herein.

The invention can be practiced with a wide variety of active agents or diagnostic agents. The drug or diagnostic agent is preferably present in an essentially pure form, is either fairly or poorly water soluble, and is dispersible in at least one liquid medium. By “fairly water soluble” it is meant that the drug or diagnostic agent has a solubility in water of greater than 30 mg/ml. By “poorly water soluble” it is meant that the drug or diagnostic agent has a solubility in water of less than about 30 mg/ml, preferably less than about 10 mg/ml and more preferably less than about 1 mg/ml.

The active agent can be selected from a variety of known classes of drugs or diagnostic agents, including, for example, analgesics, anti-inflammatory agents, anthelmintics, anti-arrhythmic agents, antibiotics (including penicillin's), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

The drugs or diagnostic agents are commercially available and/or can be prepared by techniques known in the art. The active ingredient may be present in any amount which is sufficient to elicit a therapeutic effect and, where applicable, may be present either substantially in the form of one optically pure enantiomer or as a mixture, racemic or otherwise, of enantiomers.

Non-limiting examples of representative poorly water soluble drugs or fairley water soluble drugs include: albendazole alfaxalone, acyclovir, alprostadil, aminofostin, anipamil, azidothymidine, beclomethasone, belomycin, bromocriptine, busulfan, captopril, carbamazepine, cefalexin, cefluoroxime, clonidine, corticosterone, cortisol, cyclophosphamide, cyclosporin A, and other cyclosporins, desogestrel, dexamethasone, dideoxyadenosine, doxorubicin, econazole, epoprostenol, estradiol, etoposide, felbamate, glipizide, griseofulvin, ketoconazole, metronidazole, nifedipine, oxytetracycline, piroxicam, prednisone, progesterone, taxol, taxotere, testosterone, vitamin A, and vitamin E succinate.

The compositions of the present invention comprise from about 15% to about 90% by weight of a liquid volume reduction agent. The active agent is initially presented in a liquid as dispersion. The dispersion may include a suspension or a solution and the like. The liquid may be any pharmaceutically acceptable liquid such as water, alcohol, propylene glycol, glycerin, polyethylene glycol, sorbitol, among others. The liquid volume reduction agent operates to reduce the volume of the liquid dispersion comprising the active agent thereby providing a semi-solid viscous dispersion which is further processed into a solid oral rapidly disintegrating dosage form for administration. In some aspects, the amount of the liquid volume reduction agent may range from: about 10% to about 20%; about 15% to about 30%; about 15% to about 40%; about 20% to about 40%; about 15% to about 50%; about 15% to about 60%; about 15% to about 70%; about 15% to about 80%; about 20% to about 50%; about 20% to about 60%; about 20% to about 70%; about 20% to about 80%; about 20% to about 90%; about 30% to about 40%; about 30% to about 50%; about 30% to about 60%; about 30% to about 70% about 30% to about 80%; about 30% to about 90%.

The liquid volume reduction agent is any material that reduces the volume of the liquid in the liquid dispersion comprising the active agent. Some non-limiting examples include: methylcellulose, hydroxyethylcellulose, hydroxypropylcellulose, hydroxypropylmethyl-cel lulose phthalate, noncrystal line cellulose, magnesium aluminium silicate, silicon dioxide, polyvinyl alcohol (PVA), polyvinylpyrrolidone (PVP), and the like. Other examples include: polyethylene glycol,polyoxyethylene, carbomers and cellulose-based polymers, crospovidone, silicified microcrystalline cellulose, mannitol, croscarmellose, microcrystalline cellulose, calcium silicate, calcium salts such as calcium phosphate, calcium carbonate, calcium magnesium trisilicate, calcium aluminum silicate, etc.

The liquid dispersion may originally contain for example solids of about 10% or 20% or 30% or 50% or 60% or 70% or 80%. This dispersion is then contacted with a liquid volume reduction agent which facilitates the formation of a semi-solid composition by reducing the weight percentage or volume of the liquid phase. The liquid volume reduction agent for example may reduce the liquid phase of the composition by about 10%, or 20%, or 30%, or 40% or 50% or 60% or 70% or 80% or 90% either by weight or by volume. This semi-solid composition is then subjected to processes described herein such as fluid bed drying, spray congealing, fluid bed granulation, high shear granulation or forming into a suitable dosage form. One of skill in the art is knowledgeable of these processes. See for example, Remington: The Science and Practice of Pharmacy, 19^{th} Edition, Chapter 92 (1995); U.S. Pat. No. 5,392,531, WO96/09814, which are incorporated by reference herein.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

The active agent composition can be present in the rapidly disintegrating formulations of the invention in an amount of about 0.1% to about 99.9/(w/w), preferably about 5% to about 70% (w/w), and most preferably about 15% to about 40% (w/w), based on the total weight of the dry composition.

A rapidly disintegrating solid oral dosage form of the invention can be prepared by granulating in a fluidized bed an admixture comprising a liquid dispersion of active agent and at least one liquid volume reduction agent and optionally suitable excipients to form a granulate. This is followed by tableting of the granulate to form a solid oral dosage form.

Granulation of the composition can be accomplished using a fluid bed granulator or by using high shear granulation. Fluid bed drying can also be used in making dry powder for processing into a dosage formulation.

Powders for tableting can be formulated into tablets by any method known in the art. Suitable methods include, but are not limited to, milling, fluid bed granulation, dry granulation, direct compression, spheronization, spray congealing, and spray-dying. In an exemplary process, a rapidly disintegrating dosage form can be prepared by blending a nanoparticulate composition, comprising a poorly soluble active agent and at least one surface stabilizer, with at least one pharmaceutically acceptable water-soluble or water-dispersible excipient, and, optionally, other excipients to form a blend which is then directly compressed into tablets. For example, spray-dried nanoparticulate powder can be blended with tablet excipients using a V-blender, or high-shear mixer, followed by compression of the powder using, for example, an automated press, single station Korsch press, or a high-speed Fette tablet press.

In one specific aspect, the rapidly disintegrating compositions of the present invention comprise an active agent whose particle size has remained substantially the same as compared to the particle size of the active agent in the liquid dispersion prior to the addition of liquid volume reduction agent in the manufacturing process. Thus, the rapidly disintegrating compositions of the present invention comprise active agent whose particle size is substantially preserved in the final dosage form. As used herein, the phrases “substantially preserved” or “remained substantially the same” refer to the situation in which the particle size distribution remains within a 10-20% deviation. Generally, the particle size distribution is measured and or represented as a mean particle diameter of a certain % of the particles. For example, it is customary in the art to designate particle size distribution as D90 of 10 microns, which represents that 90% ofthe particles have a mean particle diameter of 10 microns or less. Substantial preservation of particle size in the composition is significant because particle size of the active agent plays a critical role for many pharmaceutical products. For example, particle size changes may affect flow properties, dissolution, and absorption among others.

Alternatively, the present invention provides a method to prepare a rapidly disintegrating composition of an active agent without substantially changing the particle size of the active agent comprising the steps of: a) preparing a liquid dispersion of said active agent; b) adding at least one pharmaceutically acceptable liquid volume reduction agent to said liquid dispersion; c) forming a semi-solid viscous composition; and d) forming rapidly disintegrating particles; and e) optionally compressing said particles into a rapidly disintegrating tablet.

In one aspect, the rapidly disintegrating particles are formed from the semi-solid viscous composition by way of freeze-drying. The technique for freeze-drying is generally known in the art. In an alternate aspect, the particles may be formed by vaccum drying. However, vaccum drying and freeze-drying techniques should be employed where no damage to or degradation of the active agent is known to occur. Another possible technique is by way of filtration. Commonly used filtering materials may be employed. Again, in all these cases, particle size of the active agent is substantially preserved.

The rapidly disintegrating solid formulations of the invention can be in the form of tablets for oral administration. Preparation of such tablets can be by pharmaceutical compression or molding techniques known in the art. The tablets of the invention may take any appropriate shape, such as discoid, round, oval, oblong, cylindrical, triangular, hexagonal, and the like. The tablets may be coated or uncoated. If coated they may be sugar-coated (to cover objectionable tastes or odors and to protect against oxidation) or film coated (a thin film of water soluble matter for similar purposes).

In one specific aspect, the rapidly disintegrating formulations of the invention can be prepared without using a lubricating agent in a substantial amount. For example, the rapidly disintegrating tablets may be prepared wherein commonly used lubricants such as magnesium stearate are not substantially present or needed. As used herein, the term “substantially” means “to be effective.” For example, as used in the context of lubricating agents, the rapidly disintegrating tablets of the present invention may be prepared wherein a lubricating agent such as magnesium stearate is either not present or if present is not in an amount to be effective to perform its intended function.

The present invention provides a method of treating a mammal including a human, requiring the rapid availability of an active ingredient. The administered rapidly disintegrating compositions of the invention rapidly release an incorporated active agent resulting in fast onset of activity.

In general the compositions of the invention will be administered orally to a mammalian subject in need thereof using a level of drug or active agent that is sufficient to provide the desired physiological effect. The mammalian subject may be a domestic animal or pet but preferably is a human subject. The level of drug or active agent needed to give the desired physiological result is readily determined by one of ordinary skill in the art by referring to standard texts.

The following examples are given to illustrate the present invention. It should be understood, however, that the invention is not to be limited to the specific conditions or details described in these examples. Throughout the specification, any and all references to a publicly available documents are specifically incorporated into this patent application by reference.

### EXAMPLE 1

### Preparation of a Rapidly disintegrating Dosage Form of Drug A

Drug A is a poorly soluble steroidal active agent. Crospovidone, mannitol and silicon dioxide each of approximately 14% by weight are used as liquid volume reduction agent. These are blended in a double cone blender until the mixture is uniform. Drug A is suspended in water at 28% w/w. The liquid volume reduction agent mixture is then slowly added to the Drug A suspension under continuous low shear mixing to form viscous semi-solid composition. The material is then cooled to about −50° C. for about an hour. The material is then dried in a freeze drier for about 5-6 hours. Then the material is slowly brought to room temperature. This formed material is then collected in blister packs. The formulation disintegrates in about 40 seconds. The disintegration time is measured using both conventional USP apparatus and also using a stop watch and visual observation.

### EXAMPLE 2

The semi-solid material as described in Example 1 is prepared. The material is then dried as in Example 1. The dried material is then sieved using a sieve of desired size (size #16). The dried sieved material is then compressed into rapidly disintegrating tablets. The tablets disintegrate in about 30 seconds.

### EXAMPLE 3

The Example 2 above is followed to prepare dried sieved materials. The material is compressed into rapidly disintegrating tablets, but this time without using any lubricating material. The tablets disintegrate in less than 20 seconds.

### EXAMPLE 4

The procedure described in Example 1 is followed with the exception that crospovidone at 15% w/w is used as the liquid volume reduction agent. The formed material disintegrates in about 20 seconds.

### EXAMPLE 5

The Example 2 procedure is followed except that microcrystalline cellulose (about 20%) and mannitol granules (about 40%) are used in preparing granules. Crospovidone (about 6%) is used as a disintegrant. The compressed tablet disintegrates in about 35 seconds.

### EXAMPLE 6

Example 1 is followed except that croscarmellose sodium (about 5%) and silicified MCC (about 21%) are used as liquid volume reduction agents. The formed composition disintegrates in about 25 seconds.

### EXAMPLE 7

Example 6 composition is used to granulate, using mannitol about 57% as flowability agent and/or disintegrant. The compressed tablet disintegrates in about 40 seconds.

### EXAMPLE 8

Example 1 procedure is followed, except that the mixture of croscarmellose sodium (about 5%), silicified CMC (about 10%) and mannitol (about 10%) is used as liquid volume reduction agent. The formed product disintegrates in about 20 seconds.

### EXAMPLE 9

Example 8 material is used further to granulate using about 56% of mannitol as the flowability agent/disintegrant. The compressed tablet disintegrates in about 30 seconds.

### EXAMPLE 10

For the disintegration determination, a disintegration tester containing 710 micron sieves is used to test tablets in a 1000 ml deionized water bath at 37° C. Disintegration measurements are performed in accordance with USP 20.

### EXAMPLE 11

The methodology of Example 1 is used to prepare rapidly disintegrating itraconazole preparation. Further, itraconazole is prepared into rapidly disintegrating tablets of 50 mg, 100 mg, 200 mg and 400 mg per unit dose.

### EXAMPLE 12

The methodology of Example 6 is used to prepare rapidly disintegrating paclitaxel preparation. Paclitaxel is prepared into tablets of 10 mg, 20 mg, 50 mg, 100 mg and 200 mg.

### EXAMPLE 13

The methodology of Example 1 is used to prepare rapidly disintegrating dexamethasone preparation. Further, dexamethasone is prepared into rapidly disintegrating tablets of 1 mg, 2 mg, 5 mg, 10 mg, 25 mg and 50 mg per unit dose.

### EXAMPLE 14

The methodology of Example 7 is used to prepare rapidly disintegrating acyclovir preparation. Further, acyclovir is prepared into rapidly disintegrating tablets of 50 mg, 100 mg, 200 mg and 400 mg per unit dose.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method for preparing a solid dose rapidly disintegrating composition having a pharmaceutically active agent in a therapeutically effective amount, and a liquid volume reduction agent comprising from about 10% to about 90% by weight, said composition substantially completely disintegrating upon contact with a biological medium in less than about one minute, comprising:
providing a liquid dispersion of an active agent;
adding at least one pharmaceutically acceptable liquid volume reduction agent;
forming a semi-solid viscous composition; and
forming the viscous composition into rapidly disintegrating grannules that include active agent particles of about the same size as active agent particles in the liquid dispersion.

2. The method of claim 1, wherein the liquid volume reduction agent comprises from about 20% to about 90% by weight.

3. The method of claim 1, wherein the liquid volume reduction agent comprises from about 30% to about 80% by weight.

4. The method of claim 1, wherein the liquid volume reduction agent comprises from about 40% to about 90% by weight.

5. The method of claim 1, wherein the composition substantially completely disintegrates upon contact with saliva in less than about 40 seconds.

6. The method of claim 1, wherein the composition substantially completely disintegrates upon contact with saliva in less than about 20 seconds.

7. The method of claim 1, wherein the composition substantially completely disintegrates upon contact with saliva in less than about 10 seconds.

8. The method of claim 1, wherein the active agent is selected from the group consisting of:
a cardiovascular drug, a psychotropic drug, an antibiotic, an antiviral agent, an analgesic, an anti-inflammatory agent, a hormone, a respiratory agent, a gastrointestinal drug.

9. The method of claim 1, wherein liquid volume reduction agent is selected from the group consisting of a sugar, a carbomer, a cellulose-based polymer, polyethylene glycol, polyvinylpyrrolidone, polyvinylalcohol, polyoxyethylene copolymers, polyoxypropylene copolymers, polyethyleneoxide, and a mixture thereof.

10. The method of claim 1, wherein the composition is further processed into a dosage form selected from the group consisting of:
a directly compressed tablet; a granulated and compressed tablet; a sachet; and a formed dosage form.

11. The method of either of claims 1 or 2, wherein the composition is made by fluid bed granulation, spray drying, or high shear granulation.

12. The method of either of claims 1 or 2, wherein the active agent is in the form of crystalline particles, semi-crystalline particles, amorphous particles, or a mixture thereof.

13. The method of claim 2, further comprising adding one or more additional pharmaceutically acceptable excipients to the granules formed followed by compressing the granules to form a solid dose composition.

14. The method of claim 1, wherein the active agent is a poorly soluble drug.

15. The method of claim 1, wherein the active is a fairly soluble drug.

16. The method of claim 1, wherein said forming of rapidly disintegrating particles is achieved by subjecting the semi-solid viscous composition to a process selected from the group consisting of: fluid bed drying, freeze-drying; vaccum drying, spray congealing, and filtration.
